(19) United States
(12) Patent Application Publication
(10) Pub. No.: US 2021/0290502 A1
(43) Pub. Date: Sep. 23, 2021

(54) COSMETIC PRODUCT WITH A HIGH CONCENTRATION OF METAL IONS WITH SIMULTANEOUS INCORPORATION OF A PIECE OF WHITE QUARTZ AND WATER OF HIGH METAL IONS CONTENT AND METHOD FOR THE PRODUCTION THEREOF

(71) Applicant: ANAS COMPANY B.V., Amsterdam (NL)

(72) Inventors: Ioannis MOURTZINOS, Thessaloniki (GR); Aikaterini VASILATOU, Nea Smirni Attikis (GR); Vasileios ROUMELIOTIS, Tripoli (GR); Anas PUTRISS, Amsterdam (NL)

(21) Appl. No.: 17/265,248

(22) PCT Filed: Aug. 1, 2019
(86) PCT No.: PCT/GR2019/000055

§ 371 Date: Feb. 2, 2021

(30) Foreign Application Priority Data
Aug. 2, 2018 (GR) 20180100358

(51) Int. Cl. A61K 8/25; A61K 8/96; A61Q 19/00; A61K 33/00; A61K 35/02

(52) U.S. Cl. CPC A61K 8/25; A61K 8/965; A61K 2800/28; A61K 33/00; A61K 35/02; A61Q 19/00

(57) ABSTRACT

Cosmetic product with a high metal ion concentration which is produced by incorporating in the mass of the cosmetic a piece of white quartz of a specific weight and by simultaneously enriching the formula of the cosmetic product with water of high metal ions content. The final product has specific specifications based on values: conductivity (in μS/cm), pH and ion concentration. The production method can be used to increase the content of metal ions of food, food supplements, facial and body care products as well as pharmaceutical products.

### TECHNOLOGICAL FIELD

The present invention relates to cosmetic products which are produced with the use of a novel method which achieves the increased concentration, beneficial to skin health, of metal ions. The increase in the concentration of metal ions is achieved by incorporating in the mass of the cosmetic piece of white quartz of a specific weight and by simultaneously enriching the formula of the cosmetic product with mineral water with a high content of metal ions.

### TECHNICAL BACKGROUND

Until now, no such product has been known to have been produced using this particular method.

In particular, the white quartz consists of silicon oxide (SiO₂) and other metal ions. According to the scientific literature (Carretero & Pozo, 2010), metal ions protect the skin by creating a protective film when they come into contact with the skin. They also show antiseptic activity by limiting the amount of available water resulting in the creation of an unfavorable environment for the development of microbial flora. The use of analogous metal oxides as cosmetic ingredients is known. The titanium dioxide is used as a sunscreen ingredient as it reflects the ultraviolet radiation.

Mineral waters are used for their healing properties. An example of such type of mineral water is the mineral water of the Bourboule, region of France. The springs of the area are characterized as healing springs. The natural path of water, between volcanic stones, enriches the water in minerals and trace elements which contribute significantly to its healing properties. The water is rich in ions such as silicates. In particular, its physicochemical properties are: silicon ions concentration 40-100 SiO₂ mg/kg, calcium concentration 4-10 mg/kg and pH 4.5-6.5.

The use of metal ions in medicines and cosmetics is widespread, as they constitute bioactive ingredients (ingredients with beneficial properties). As an example, the zinc oxide compounds (ZnO) create a protective layer against environmental factors. Furthermore, the compounds have antiseptic activity, while the same applies with sulphide compounds of aluminum and copper. Metallic elements originating from minerals are also used to combat gastroesophageal diseases and disorders, such as compounds of aluminum and magnesium silicates, and calcium carbonates derivatives. Other examples are magnesium compounds that are introduced into cosmetic creams and powders, but also in bath salts, which they mainly derive from silicates or sulphurous minerals. In addition, calcium compounds are used in toothpaste formulas mainly due to their bleaching properties.

A major disadvantage of the use of metal ions and their compounds in products is that said use takes place through the creation of different formulations such as emulsions, creams and dry powders. The isolation of metal ions from their natural sources through physicochemical processes shall precede. Such processes are the effect of energy in the form of ultrasounds or heat, and the creation of dispersion systems with the use of solvents. All these methods include energy-intensive processes and the use of additional chemicals (excipients) in order for the processes to be stabilized, a fact which vertically increases the cost of the production process.

On the contrary, with the present invention the above disadvantages are eliminated. As no prior receipt of mineral ingredients from the quartz or the use of a special formulation, the preparation of which includes both physical and chemical processes, is required, since it includes only natural sources and natural processes. Moreover, the use of organic solvents, and energy in any form, is avoided, a fact which makes the production method of the invented products environmentally friendly and ecological. In addition, the invented method allows the use of different types of minerals and different types of mineral waters and it constitutes the simplest method of enrichment of cosmetics with metal ions, where no high temperatures, ultrasounds or microwaves are used, methods that require energy consumption and vertically increase the production cost, while the final product has specific specifications regarding pH, conductivity and metal ion concentration.

Finally, the process of transferring metal ions from the white quartz pieces to the mass of the cosmetic, is an active process that takes place throughout the life of the cosmetic in its packaging and the proposed method does not affect the pH of the product which is a critical physicochemical parameter of a cosmetic.

The confirmation of the transfer of metal ions from the white quartz piece to the cosmetic system is done by measuring the physicochemical data, namely the concentration of cations, the pH and the conductivity.

Therefore, the basic purpose of the present invention is the production of cosmetic products such as cleaning products, creams and sunscreens, with a high concentration of metal ions which is achieved by incorporating a white quartz piece into each packaging of the cosmetic product and by enriching the formula of the cosmetic product with mineral water which is carried out during the aqueous phase of preparation of the cosmetic product. In this way, metal ions are transferred from the surface of the white quartz to the mass of the cosmetic product throughout the life of the cosmetic product.

### DISCLOSURE OF THE INVENTION

In order to make our invention understood by those skilled in the art, the method of incorporating the white quartz into the mass of the cosmetic product and of enriching it with mineral waters is described below.

The process starts with the selection of the appropriate white quartz stones, which originate from the island of Madagascar. This process involves extracting them from the ground, cutting them to the desired size, and their treatment which includes their mixing with natural abrasives such as sand and silicon carbide in order to make them smooth and rounded, and, finally, their cleaning which is made by washing them with water.

Subsequently, a piece of white quartz of a specific weight is inserted into each piece of cosmetic product during the packaging stage of the product, to a white quartz piece content from 2% w/w to 4% w/w of the total mass of the product.

The enrichment of the cosmetic with mineral water takes place by adding the mineral water of suitable specifications during the aqueous phase of the cosmetic production to a specific content from 2% w/w to 10% w/w of the total mass of the product. In this way, it is possible to strengthen the products with metal ions, ultimately aiming at their transferring to the skin. The beneficial ions protect the skin by creating a “membrane” that promotes the defense against natural and chemical factors.

The confirmation of transfer of metal ions from the piece of white quartz to the cosmetic system is achieved by measuring the physicochemical values: cations concentration (Na⁺, K⁺ Ca⁺², Mg⁺²) and conductivity in aqueous solution to which a piece of white quartz (Sample 2) has been added and the values are compared with the measurements values of the sample which does not include quartz (Sample 1). The measurements take place over a period of fifteen days following the preparation of the solutions.

| Parameter | Sample 1 | Sample 2 |
|---|---|---|
| Conductivity (μS/cm ) | 322 | 330 |
| Na⁺ Concentration (ppm) | 56 | 58 |
| K⁺ Concentration (ppm) | 40 | 42 |
| Ca⁺² Concentration (ppm) | 0.8 | 1.6 |
| Mg⁺² Concentration | 1.8 | 2.6 |
| (ppm) | | |
| Cl⁻¹ Concentration (ppm) | 5.5 | 6.9 |

The following examples are given in order to explain the present invention:

### EXAMPLE 1

5% (w/w) of mineral water is added during the aqueous phase of production of the cosmetic product. A piece of white quartz weighing 4 g is added to a 100 g packaging of cosmetic product. Its quality characteristics are defined by the values in Table 1.

TABLE 1

| Parameter | Value |
|---|---|
| Conductivity (μS/cm) | >300 |
| pH | <5.7 |
| Na⁺ Concentration (ppm) | 58 |
| Na⁺ Concentration (ppm) | 42 |
| Ca⁺² Concentration (ppm) | 1.6 |
| Mg⁺² Concentration (ppm) | 2.6 |

### EXAMPLE 2

8% (w/w) of mineral water is added during the aqueous phase of production of the cosmetic product. A piece of white quartz weighing 6 g is added to a 100 g packaging of cosmetic product. Its quality characteristics are defined by the values in Table 2.

TABLE 2

| Parameter | Value |
|---|---|
| Conductivity (μS/cm) | >350 |
| pH | <5.7 |
| Na⁺ Concentration (ppm) | >58 |
| Na⁺ Concentration (ppm) | >42 |
| Ca⁺² Concentration (ppm) | >1.6 |
| Mg⁺² Concentration (ppm) | >2.6 |

The method described above can be used for the enrichment of cosmetic products for the care of the face, body and hair as well as for the enrichment of pharmaceutical products.

1. Cosmetic product with a high concentration of metal ions with simultaneous incorporation of a piece of white quartz and water of high metal ions content characterized in that, it contains, per individual packaging, a piece of white quartz in quartz/mass ratio from 2% w/w to 4% w/w, resulting in the transfer of the metal ions from the white quartz surface to the mass of the cosmetic product throughout the life of the product and mineral water with the desired specifications at a content from 2% w/w to 10% w/w of the total mass of the product.

2. A method of production of a cosmetic product with high concentration of metal ions with simultaneous incorporation of a piece of white quartz and water of high metal ions content, according to claim 1, characterized in that in the first stage of the method a treatment of the white quartz which is mixed with natural abrasives, such as sand and silicon carbide, takes place, in order for the stones to become smooth and of defined shape.

3. A method of production of a cosmetic product with high concentration of metal ions with simultaneous incorporation of a piece of white quartz and water of high metal ions content, according to claim 1, characterized in that, in the second stage of the method the treated white quartz pieces are added to each individual packaging of cosmetic product in a quartz/product mass ratio from 2% w/w to 4% w/w.

4. A method of production of a cosmetic product with high concentration of metal ions with simultaneous incorporation of a piece of white quartz and water of high metal ions content, according to claim 1, characterized in that, during the aqueous phase of production of the cosmetic, mineral water of appropriate specifications is added to the formula of the cosmetic at a specific content: from 2% w/w to 10% w/w.

5. A method of production of a cosmetic product with high concentration of metal ions with simultaneous incorporation of a piece of white quartz and water of high metal ions content, according to claim 1, characterized in that, the confirmation of transfer of the ions from the piece of white quartz is made through the measurement of the conductivity and of the cations in an aqueous solvent and comparison is made with the respective values of a solvent where no quartz is added.

6. A method of production of a cosmetic product with high concentration of metal ions with simultaneous incorporation of a piece of white quartz and water of high metal ions content, according to claim 1, characterized in that, in the final stage a measurement of the conductivity (in mS/cm), of the pH and of the ion concentration is made in order for the final product to meet the desired specifications.

7. A method of production of a cosmetic product with high concentration of metal ions with simultaneous incorporation of a piece of white quartz and water of high metal ions content, according to claim 2, characterized in that, it can alternatively be used for the production of food supplements and pharmaceutical products.
